(19) United States
(12) Patent Application Publication
(10) Pub. No.: US 2013/0025593 A1
(43) Pub. Date: Jan. 31, 2013

(54) Dry powder inhaler

(71) Applicants: Aparna Thirumalai Anandampillai, Chennai (IN); Anand Vishnu Thirumalai Anadampillai, Chennai (IN); Vijayan Thirumalai Anadampillai, Chennai (IN)

(72) Inventors: Aparna Thirumalai Anandampillai, Chennai (IN); Anand Vishnu Thirumalai Anadampillai, Chennai (IN); Vijayan Thirumalai Anadampillai, Chennai (IN)

(21) Appl. No.: 13/507,418

(22) Filed: Jun. 25, 2012

(30) Foreign Application Priority Data
Dec. 30, 2009 (IN) 3243/CHE/2009

(51) Int. Cl. A61M 16/10

(52) U.S. Cl. 128/203.15

(57) ABSTRACT

It has a complex shaped hollow body with front mouthpiece (2) covered by dust cap. Drug strip (1) is held in channel (7) with a drug hole (6). Handle (4) with a sharp piercing rod (5) moves on a pivot (10) at the body back. The pierced powder falls through drug hole (6) into drug chamber (23) with a bottom sliding drug release plate (11) having a knob (8), a spring (14) enclosed in sleeves (15,17) and having a drug hole (12). The hollow body has the back air hole (3) with a dust filter (22), then narrows to a jet hole (19) & continues as hollow mouthpiece. A baffle mesh (16) breaks falling powder into a spray. To use, drug strip (1) is pierced by handle (4), inhaler kept in mouth, and knob (8) pressed and inhaled. Plate (11) slides drug hole (12), drug falls on baffle (16), air jet breaks and forms drug spray for more lung deposit. For drugs in capsule, the inhaler is modified with middle placed capsule (26) with a back piercing rod (25).

### TECHNICAL FIELD

This invention in general relates to medical equipments, specifically a novel kind of deep inhaling lung physiotherapy device and low mouth coating dry powder inhaler.

### STATE OF ART

No inhaler is now used as safe pure air breathing lung physiotherapy device. Yoga-like deep breathing techniques provides asthmatics relief too, by improving the elasticity of lung. Asthmatics use metered dose pressure inhalers that send a spray of drug with evaporating propellant (Ozone depleting fluorocarbons) or a dry powder inhaler (DPI) that has no propellant. This device belongs to dry powder inhaler (DPI) type. Existing DPI inhalers are single dose (rotahalers) or a multidose (Turbuhalers, Discus, disc inhalers) with drugs as powders inhaled in a plastic body with a short and wide mouthpiece that wastes the drug, as mouth coating 90% with only 10% drug deposit in lungs, poor spray creation with large clumps with large oral deposit that needs water to swallow. The following description gives critical examination of the inhalers known in the art with its shortcomings. Further in order to overcome the problem associated with prior art inhalers, the invention offers the solution to overcome the impediments in the construction and the process of using the inhaler.

Defects in the existing DPI inhaler devices are as follows:

1. Are just inhalers, with no lung exercising option.

2. If inhaled, effort & depth is low, repeated doses needed, wasting drug.

3. Its wide mouth gives short 2-3 seconds for inhalation!

4. The drug spray is into mouth and little to lungs.

5. Spray mist is large clumps, not fine uniform spray

6. Dusty, polluted air is inhaled without a filter that may cause disease to users!

7. With a little tilt, powders fall off the inhalers before inhalation-drug wastage.

The invention is a further improvement of our earlier invention WO2008/139490 which is larger, uses more plastic, with a different drug release & spray formation. The new inhaler is smaller, easy to hold, use with a better uniform spray. Spray mechanism is a new linear single axis air jet helped drug mist. The pistol shaped body was eliminated, the drug release mechanisms was simplified, kids & illiterates will easily use. These improvements needed further research.

An extensive search has been carried out using the Internet and related patent specifications were studied for low mouth coating with uniform spray and easy inhalation. Since the present invention is radically different in a better easy inhaling spray with no extra energy, the inventor is unable to site any patent specification out of the available databases except

WO 2010039202 of LEWIS SCOTT is a dry powder inhalers with rotatable piercing mechanisms facilitate the use of dose container assemblies having dose containers arranged in concentric rows.
US 2009320837 of SMITH SIMON is inhaler comprises a cyclone chamber having a cyclone chamber outlet extending axially
RU 2141849 MARK BERNARD has impeller positioned eccentrically in mixing chamber. Impeller is rotated by motor at high speed. Plunger feeds dose of medicinal powder into chamber so that all particles of powder are available for mixing
U.S. Pat. No. 7,617,822 of De Boer A breath actuated dry powder inhaler, comprising a substantially disc shaped air circulation chamber for de-agglomeration of entrained powdered medicament using the energy of the inspiratory air stream
U.S. Pat. No. 6,971,384 of Gieschen A dry powder inhaler has beads containing dispersion chamber
US patent 2002170560 of YOUNG MATTHEW is a blister disc with a complicated dispersion mechanism and a short mouth piece
US patent 2004069303 of BROWN DAVID is a multi dose powder chamber with a rotating disc feed. The angled air flow jet is after the feed, a poor spray forming mechanism in a short mouth piece. The jet is not at powder feed area but down stream.
US patent 2004163644 of GIESCHEN ANDREW W has a chamber with beads for deagglomeration of powder
WO02053215 of GENOVA PERRY is for a single dose inhaler with a complicated seal plate that vibrates for spray formation.
U.S. Pat. No. 6,427,688 of Ligotke is for single dose bead assisted deaggregation of powder spray formation
U.S. Pat. No. 5,513,630 of century has a gas with actuator for spray
U.S. Pat. No. 6,003,512 of Gerde uses the forces of pressurizing and depressurizing gas loaded into powder agglomerates located in an enclosed powder chamber for spray formation.
U.S. Pat. No. 6,142,146 of Abrams is for an electronic vibrator for spray formation.
U.S. Pat. No. 6,116,238 of Jackson is a slider mechanism to unseal the blister pack
U.S. Pat. No. 6,029,663 of Eisele has a carrier disk with a blister shell sealed by a shear layer. A tab is adhered to the shear layer, underneath the blister shell. An actuator pushes against the tab, causing the shear layer to tear away, releasing the powder drug contents from the blister into the dry powder inhaler.
U.S. Pat. No. 5,921,237 of Eisele is an inhaler with a blister disc with an actuator. A switch senses pressure in the mouthpiece and switches on a motor spinning an impeller within the aerosolizing chamber. The impellor also is coated with the powder in use decreasing dose.
U.S. Pat. No. 5,921,237 of Vaghefi uses pressurised gas to burst the powder pack. An electrostatic charge for spray formation in a nonstick lining chamber is costly too.
U.S. Pat. No. 5,577,497 of Mecikalski has a motor spinning the impeller at high speed & sprays powder released by a plunger into the chamber so that all powder particles are aerosolized to form a fine, low-density, low velocity, dry mist.
U.S. Pat. No. 5,694,920 of Abrams has a piezoelectric vibrator 54 for vibrating a diaphragm to a powder, and a controller 70 for controlling vibration so as to suspend a portion of powder in a fluidized state. An electrostatic charge plate 32 draws powder of selected particle size into the inhalation stream.
None of these use up the force of inhalation to create a high velocity jet that forms drug spray. All these inhalers are focused on powder spray creation, not the easy inhalation part We inhale 500 cc in adults—smaller in lung diseases. This volume must be used to form a spray and send to air sacs for blood transfer. Ideal inhalation is by breathing through a small 5-8 mm diameter mouthpiece which creates higher velocity air jet from even 300 ml of air inhalation that is used for spray formation as in our device. All spray produced if not properly inhaled will deposit in mouth only leading to failed low lung deposit, as in all devices.

Further the invention is addressed to the process of using the new inhaler, which is unique in design and construction, working, use and different with a lower mouth coating mechanism which helps to deposit the drug only in the lungs correctly.

Further objects of the invention will be clear from the ensuring description.

### SUMMARY OF THE INVENTION

The inhaler comprises of an easy holding complex hollow plastic body with a front narrow longer mouth piece, a broader back cylindrical part with a dust filter and the middle drug dropping part. The needed powder is packed as a blister strip and held in strip channel on top of the body. A sharp pointed piercing rod of a pressing handle pivoted on the outside of the cylindrical body pierces the strip to deposit the dry powder in a drug chamber in the body. This small chamber 3-5 mm has a drug release plate with a knob to drop the powder in the spray zone. The outside air is pulled through the dust filter fitted in the body for clean air inhalation. The air is now channeled as a 3-5 mm fast jet into which the powder falls, forms an instant spray of powder. A baffle breaks any clumps to uniform spray. The longer mouth piece leads the spray straight to windpipes for lung deposit.

### STATEMENT OF DRAWINGS

These and other objects and features of the invention will become more apparent upon perusal of following description taken in conjunction with accompanying drawings wherein:

FIG. 1 shows the novel inhaler.

FIG. 2 shows the exploded view of the novel inhaler a closed .B open hole 16

FIG. 3 A, B shows mode of use

FIG. 4 shows the use and spread of the drug mist beyond throat.

FIG. 5 shows the conventional inhaler, (a) single dose, (b) multi dose

FIG. 6 shows a modification for capsule piercing inhaler

### DESCRIPTION OF PREFERRED EMBODIMENTS

The following specification describes salient features of invention, the method of construction, the method of use and the advantages of the novel invention.

The novel inhaler has an easily held complex shaped plastic body for easy holding with a middle drug part, back air filtering and a front mouthpiece parts. The back of the inhaler is a cylindrical body with a large air intake hole with a dust filter. The outside of this cylindrical body has a pivot with a pressing handle provided with a front piercing pointed rod. This rod sits on a drug strip held on a strip channel with a hole. The hole drops the powder to a short drug chamber having a lower sliding plate with a knob. The knob releases drug as needed in inhalation. As the powder falls on fast moving inhaled air jet, it is converted to a spray, flows in the longer mouthpiece beyond the tongue to wind pipes for lung deposit. The needed drug is packed as a long strip of blister pack. The novel inhaler according to the invention is loaded with drug strip, punctured and kept in mouth, air is sucked in through the mouthpiece, press the knob to release the drug. Drug is now released as a soft spray which travels to lungs for better effect.

The novel inhaler according to invention is better because of deeper clean air inhalation. The long slender mouth piece releases drug spray into wind passages without mouth coating unlike the short mouth pieces of existing inhaler.

The conventional single dose existing powder inhaler FIG. 5A consists of a transparent body with an air inlet at the top in which the drug capsule (1) is placed. The body has at the other end has a wide short [1.5 cms], tapering mouthpiece (2). To use, the drug capsule is fitted and twisted to break the capsule. The mouth piece (2) is kept in mouth. Air is inhaled. Inhalation is shallow and fast as the mouthpiece area is large with large mouth coating. The device has not been improved for decades. In FIG. 5B is a multi dose inhaler with a box having the disc (1) with many doses. The disc is pierced by a needle to fall on an area near the short mouth piece (2) for a poor shallow inhalation. None has the air dust filter

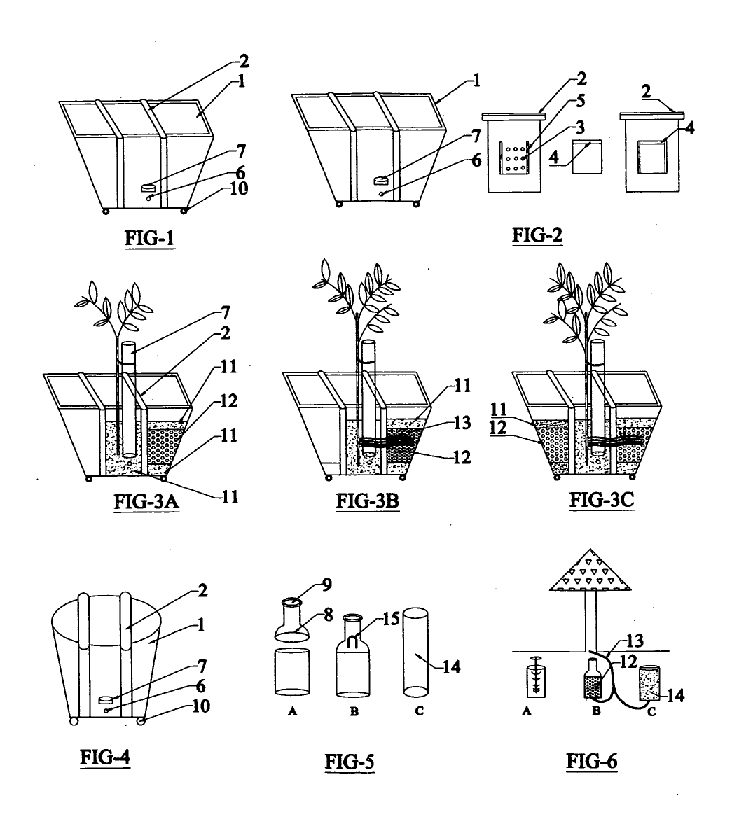

The new inhaler FIG. 1-4 comprises of a complex shaped hollow body preferably of plastic or metal. It has a drug strip (1) with needed drugs placed in an upper channel (7). The drug strip is pierced by an upper short sturdy sharp pointed rod (5) that is fitted in a pressing handle (4) working on a pivot (10) fitted to outside of the body for easy pressing. The drug falls through a hole (6) ) to a drug chamber (23) of adequate powder volume (larger for macro, smaller for micro doses) and with a bottom hole for drug. There is a sliding drug releasing plate (11) with a knob (8) with a spring (14) enclosed in bigger (15) and smaller (17) sleeves for smooth play of spring. The plate has a hole (12) to drop the drug into lower air channel. The drug hole (12) is closed usually. When the knob (8) is pushed, the spring is compressed, the plate moves in the slot (9), the plate hole (12) is below the drug chamber & allows drug to drop into air channel of mouth piece (2). The drug release plate moves in the slot (9) of the drug part of the body. The small projecting back clips (13) of the drug release plate keeps it in the slot (9). The drug plate has a terminal split (18) for inserting the plate in slot. The slot (9) just fits the plate (11). The body has a large back air hole (3) with a dust filter (22) at the back or on the side of the body. The dust filter traps air dust for clean air inhalation, not present in existing inhalers. Body at the back, is at least 20-30 mm in diameter for adults and tapers smoothly to a cone (20) and then to a narrowing 6-8 mm air jet hole (19) channel forming a high velocity novel jet that sprays drug as a mist. Mesh baffle (16) breaks the clumps of powder. This larger back 20 mm air channel, narrowed to a 6-8 mm air channel generates a high velocity air flow that sucks the released drug, sprays the drug as a thin uniform narrow spray and is new. The mouthpiece (2) is shaped as a long narrow tube at the front of the body for 3-5 cm. A dust cap fits mouthpiece for clean storage. For kids the back air inlet may be 15-20 mm in diameter.

The device gives longer, deeper inhalation, does not spray into mouth, directs flow of mist maximally to air passages delivering correct doses, and is safe for children, aged, even in disorientation. Without drug, the device can improve the lung function as deep breathing physiotherapy device.

To use, the novel inhaler, the drug blister pack (1) is mounted in the drug channel 7; the blister is pierced by the pointed tipped rod (5) by pressing the handle (4). The powder (21), FIG. 3A falls into the drug chamber (23) through upper hole (6). Mouthpiece is kept between the lips and sucked, FIG. 4. The back air hole (3) with the dust filter allows clean air to be sucked in inhalation! The drug release knob (8), FIG. 3B is pressed in early inhalation, compressing spring (14) moving the trap plate, allowing the fall of drug through hole (12) of trap plate in to the air jet below. Drug powder is sprayed as it falls on the mesh baffles (16) by air passing back to front through the mouthpiece (2). The mouthpiece is longer and projects into the mouth as in FIG. 4 producing a mist directed to the windpipe and not into mouth. The drug is carried to distal air passages uniformly, because of deep and slow inhalation. The increased duration (2-3 seconds longer) and deeper inhalation also helps in spread of mist. The drug is delivered better maximally, with lower mouth coating. Releasing the knob (8) makes the spring slide the plate back and stops the drug fall and spray. The remaining powder can be inhaled in next inhalation. Linear drug strip easily shows doses left without need of counters.

The inhaler is made of plastic with the orifices, mouth piece, air & drug chambers incorporated as a unit or as separate segments easily assembled. The inhaler parts are moulded in dies with suitable cavities. It can be used as clean air deep breathing physiotherapy unit with no drug used for improving lung elasticity by breathing through many times a day. This practice improves lung elasticity too. For children smaller inhaler dimensions are used

The device can be modified. The mouthpiece tube is made as two pieces adjusted on a screw or sliding mechanism for varying the length. Electronic sensing spraying and counting are possible, but will make the device costly and heavy. The body may be a transparent plastic. Various shapes for body [e.g. oval, hexagonal] and divergent mouthpiece [hexagonal] may be used. Any other design for holding and easy pressing can be used. Air hole (3) may be on the side or back of the body. Dose available or used is indicated by printing the blister pack. The drug strip may be folded on a ring and rotated by a knob for feeding the drug channel. The drug strip may be made as a cassette and fitted to channel or a circular blister pack with a handle with a pointed piercing rod, a rotating base for rotating the drug blister to feed the drug chamber with. The drug chamber may be smaller for micro doses of powder. Instead of knob pressing, spring loaded drug release in inhalation due to suction can be made, the suction, releasing drug from chamber through a spring, sliding the hole (12) below the drug chamber.

In FIG. 6 is an inhaler with drug in a capsule. The back air inlet (3) has the filter. A capsule (26) is loaded by opening the plate (28) at the middle. The mouthpiece (2) with a mesh baffle (16) forms the front part. the capsule is pierced by a pointed needle (25) pushed by a push button (24) with an inside spring (14). The capsule (26) is loaded inside the inhaler, the push button is pierced, the needle pierces the capsule at both ends and bounces back by the recoil of spring forming holes (27). The inhaler is kept in mouth &inhaled, the drug is pulled through the hole of the capsule with a soft spray. The capsule is removed and new capsule loaded by opening the middle refitting plate (25).

The term “drug formulation” means active drug (or a physiologically acceptable salt thereof) optionally in combination with one or more other pharmacologically active agents such as anti-inflammatory agents, analgesic agents or other respiratory drugs and optionally containing one or more excipients. The term “excipients” as used herein means chemical agents having little or no pharmacological activity (for the quantities used) but which enhance the drug formulation or the performance of the system. For example, excipients include but are not limited to surfactants, preservatives, flavorings, antioxidants, and antiaggregating agents.

Drug formulation for use in the invention may, if desired, contain one or more other pharmacologically active agents, selected from any suitable drug useful in inhalation therapy. Medicaments may be selected from, for example, sildenafil for pulmonary hypertension, analgesics, e.g. codeine, dihydromorphine, ergotamine, fentanyl or morphine; anginal preparations, e.g. diltiazem; antiallergics, e.g. cromoglycate, ketotifen or nedocromil; antiinfectives e.g. cephalosporins, pentamidine; antihistamines, e.g. methapyrilene; anti-inflammatories, e.g. beclomethasone, fluticasone propionate, flunisolide, budesonide, tipredane or triamcinolone acetonide; antitussives, e.g. noscapine; bronchodilators, e.g. salbutamol, salmeterol, ephedrine, adrenaline, fenoterol, formoterol, isoprenaline, albuterol, metaproterenol, phenylephrine, phenylpropanolamine, pirbuterol, reproterol, rimiterol, terbutaline, isoetharine, tulobuterol, orciprenaline, or (-) -4-amino-3,5-dichloro-.alpha.[[[6-[2- (2-pyridinyl) ethoxy]hexyl]amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon, leuprolide and genetic fragments or anti cancer drugs or vaccines for influenza or any such lung absorbable drugs. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament and/or to minimize the solubility of the medicament.

Drug formulations for Asthma may contain fluticasone propionate in combination with a bronchodilator such as salbutamol (e.g. as the free base or the sulphate salt) or salmeterol (e.g. as the xinafoate salt) as a 5 micron powder. Combinations for the other diseases as sildenafil for pulmonary hypertension, insulin for diabetes, luprolide for prostrate cancer etc may be used for treatment.

The particle size of the particular (e.g., micronised) drug should be in the range of 1-10 microns, e.g., 2-5 microns.

It will be apparent to those skilled in the art that modifications to the invention described herein can readily be made without departing from the spirit of the invention. Protection is sought for all the subject matter described herein including any such modifications.

### ADVANTAGES OF THE NEW INVENTION

1. Slow, long and deep inhalation, as air is drawn through a slender mouthpiece.

2. There is less mouth coating and waste of drug.

3. Useful for kids, elders who can now inhale easily.

4. The filter removes all air polluting dust and germ particles for safe inhalation.

5. The air inlet, mouth piece, spray jet are in linear axis for easy soft inhalation.

6. Compact, easily held & used,

7. The drug does not fall off even with tilt.

8. Can be used as deep breathing physiotherapy unit with no drug used.

We claim:

1) An improved dry powder inhaler where in the improvement is a complex compact hollow body with better drug spray creating air jet channel, the said inhaler comprising a complex shaped hollow body with a back air inlet, a middle drug part and a front slender mouthpiece, the said drug part having a channel (7) with the drug blister strip (1) on the top surface, a pressing handle (4) with a short strong sharp piercing pointed rod (5) to puncture the said drug blister, the said handle moving in a back pivot (10) at the back of the outside of the said body, the said powder of the punctured drug blister falling through a hole (6) into a short drug chamber (23), the said drug chamber has a lower sliding drug releasing plate (11) moving in a slot (9) in the body, the said drug releasing plate having a pressing knob (8), a spring (14) around the neck of the knob, a drug dropping hole (12), and held in the said body slot (9) by a clip end (13), the said body having a large back air intake hole (3) with a dust filter (22) leading to the hollow inside on back side, the said body at the front has a hollow long narrow mouthpiece with an inside powder declumping baffle mesh (16), the hollow inside is one continuous path from the wide back air inlet, narrowing in middle forming a jet hole (19), receiving the drug and continues as front hollow mouthpiece, the drug as it is dropped in air channel forms a spray that is sprayed through the mouth piece (2) into wind passages, such that the said drug spray with the inspired air flows into the lungs,

2) An improved dry powder inhaler as claimed in claim 1, wherein the body has on the top surface a handle (4) with a front short sharp drug blister piercing pointed tipped rod (5) and moving in a back pivot (10) attached to back end of the outside of the said body, the said handle (4) is pressed to pierce the drug blister strip (1) mounted on a drug channel (7) for effortless piercing and dropping the powder through the drug hole (6) into the said drug chamber (23) of the said body.

3) An improved dry powder inhaler as claimed in claim 1 wherein the said drug part of the body has a drug chamber (23) with a top drug hole (6) for the pierced powder from the said drug strip (1) held in the drug strip channel (7), the said drug chamber of needed volume has at the bottom a sliding drug releasing plate sliding in a slot (9), the said plate having a drug dropping hole (12), such that the dropped drug joins the air channel of hollow mouth piece and sprays drug powder on contact with the fast moving air jet.

4) An improved dry powder inhaler as claimed in claim 1 wherein the hollow body of 15-30 mm diameter has on its side or back has an air inlet (3) with a dust filter (22), the said hollow air path narrowing inside as cone (20) ending as a high velocity jet hole (19) of 5-9 mm diameter and then receives the drug, continues as the hollow inside of mouthpiece, the narrowed air path creating a high velocity linear air flow for spraying the powder.

5) An improved dry powder inhaler as claimed in claim 1 wherein the mouth piece (2) is a slender hollow tube lesser than 10 mm diameter, the free front mouth end having a detachable dust cap for dust protection,

6) An improved dry powder inhaler as claimed in claim 1 wherein, the said body in the middle drug part has across a slot (9) with a sliding drug releasing plate, the said plate having a back clip (13) to hold in the slot, a drug dropping hole (12), a neck with a spring (14) and a pressing knob (8) such that, pressing the knob moves the said drug release plate in the said slot (9), sliding the hole (12) below the drug chamber (23) and releasing the drug into air jet for drug spray only in inhalation.

7) An improved dry powder inhaler as claimed in claim 1 wherein, wherein the inhalation drug is the micronized single or combination of active pharmacological agents and packed as a blister drug strip, each blister fitting the drug hole (6) for easy piercing and drug dropping into the said dug chamber (23), the whole drug strip fitting the drug channel (7) of the middle drug part of the said inhaler.

8) An improved dry powder inhaler for drugs filled in capsules wherein the drug filled capsule (26) is placed in the middle part of the inhaler by opening plate (28), the inhaler has at the wide back an air inlet (3) provided with a filter and a push button (24), the push button having inside a spring (14) surrounding an inside piercing pointed tipped needle (25), the needle (25) piercing the both ends of the drug capsule with holes (27), the front mouth piece (2) having a baffle (16) such that the air from the back air inlet narrows and passes through pierced holes (27) spraying the powder through the mouth piece (2).
